(19) United States
(12) Patent Application Publication
(10) Pub. No.: US 2005/0090804 A1
(43) Pub. Date: Apr. 28, 2005

(54) Endoluminal prosthesis endoleak management

(75) Inventors: Michael V. Chobotov, Santa Rosa, CA (US); Robert G. Whirley, Santa Rosa, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TriVascular, Inc., Santa Rosa, CA (US)

(21) Appl. No.: 10/691,849

(22) Filed: Oct. 22, 2003

(51) Int. Cl. A61M 31/00

(52) U.S. Cl. 604/509; 606/200

(57) ABSTRACT

The present invention provides methods and compositions for managing endoleaks in a perigraft space around an endovascular graft. In one embodiment, a blood flow through the endovascular graft is temporarily reduced and an embolic material is delivered into the perigraft space while the blood flow through the endovascular graft is reduced. The embolic material may comprise polyethylene glycol diacrylate, pentaerthyritol tetra 3(mercaptopropionate), and a buffer.

### BACKGROUND OF THE INVENTION

The present invention relates to systems and methods for the treatment of disorders of the vasculature. More specifically, the present invention is related to management of endoluminal prosthesis endoleaks.

For indications such as abdominal aortic aneurysms (AAA) and thoracic aortic aneurysms (TAA), traditional open surgery is still the conventional and most widely-utilized treatment when the aneurysm's size has grown to the point that the risk of aneurysm rupture outweighs the drawbacks of surgery. Surgical repair involves replacement of the section of the vessel where the aneurysm has formed with a graft. It is effective in preventing death from aneurysm rupture, and its long-term efficacy is well known. An example of a surgical procedure is described by Cooley in Surgical Treatment of Aortic Aneurysms, 1986 (W.B. Saunders Company).

Despite its advantages, however, open surgery is fraught with relatively high morbidity and mortality rates, primarily because of the invasive and complex nature of the procedure. Complications associated with surgery include, for example, the possibility of aneurysm rupture, loss of function related to extended periods of restricted blood flow to the extremities, blood loss, myocardial infarction, congestive heart failure, arrhythmia, and complications associated with the use of general anesthesia and mechanical ventilation systems. In addition, the typical patient in need of aneurysm repair is older and in poor health, facts that significantly increase the likelihood of complications.

Due to the risks and complexities of surgical intervention, various attempts have been made to develop alternative methods for treating such disorders. One such method that has enjoyed some degree of success for abdominal aortic aneurysms is the catheter-based delivery of a bifurcated stent-graft via the femoral arteries to exclude the aneurysm from within the aorta. Endovascular repair of thoracic aortic aneurysms is also gaining favor as an acceptable mode of treatment.

Endovascular repair of aortic and thoracic aneurysms represents a promising and attractive alternative to conventional surgical repair techniques. The risk of medical complications is significantly reduced due to the less-invasive nature of the procedure. Recovery times are significantly reduced as well, which concomitantly diminishes the length and expense of hospital stays. For example, open surgery to repair an abdominal aortic aneurysm requires an average nine-day hospital stay and two days in the intensive care unit. In contrast, endovascular repair typically requires a two-to-three day hospital stay. Once out of the hospital, patients benefiting from endovascular repair may fully recover in two weeks, while surgical patients require at least six to eight weeks.

Despite these and other significant advantages, however, endovascular-based systems have a number of shortcomings. For example, it is estimated that at least twenty percent of all endovascular AAA repairs experience a Type I or Type II endoleak. A Type I AAA leak refers to blood flow into the aneurysm sac that is caused by the incomplete sealing of the proximal and/or distal ends of the endovascular graft against the aorta or iliac arteries. A Type II AAA endoleak refers to perfusion of the aneurysm sac via retrograde flow through a branch or collateral artery, such as the inferior mesenteric artery (IMA) or the lumbar arteries. When endoleaks occur, there is a continued, persistent flow of blood into the aneurysm sac that pressurizes the sac and leaves the patient at risk of aneurysm rupture.

Methods of treating Type I and Type II AAA endoleaks include therapies such as the introduction of coils (as described in, e.g., U.S. Pat. Nos. 4,994,069 to Ritchart, et al. and U.S. Pat. No. 6,117,157 to Tekulve), particles, or a liquid embolic material into the aneurysm sac. An illustrative example of a liquid embolic material is ethylene vinyl alcohol copolymer (EVOH) dissolved in a solvent such as a dimethyl sulfoxide (DMSO), such as that manufactured and sold under the trademark Onyx™ by Micro Therapeutics, Inc. of Irvine, Calif. and described in U.S. Pat. No. 6,203,779 to Ricci et al. Coiling of the sac branch vessels can be time consuming, costly, and may require extensive fluoroscopy time (and its concomitant undesirable radiation exposure). One problem with treating endoleaks is the possibility of distal perfusion of the embolic material away from the aneurysm sac. Such distal perfuision of the embolic material creates the potential of embolic complications in the bowels and peripheral circulation.

For the above reasons, improvements are needed to effectively manage endoleaks around an endoluminal prosthesis while minimizing the potential for undesirable distal perfusion away from the aneurysm sac.

### BRIEF SUMMARY OF THE INVENTION

The present invention provides methods, embolic materials, systems, and kits for managing endoleaks around an endovascular graft that is disposed in a diseased portion of a body lumen, such as an artery.

In one aspect, the present invention provides a method of reducing blood flow into a perigraft space between an endovascular graft and an artery wall. The method comprises accessing the perigraft space with a delivery device and delivering an embolic material into the perigraft space with the delivery device. The embolic material may comprise polyethylene glycol diacrylate, pentaerthyritol tetra 3(mercaptopropionate), and a buffer.

Individual components of the embolic material may be mixed in vitro or in vivo to create the embolic material. The buffer may include glycylglycine and may be provided in a proportion ranging from about 5 to about 40 percent weight, and preferably about 22 to about 27 weight percent. Alternatively, the buffer may comprise N-[2-hydroxyethyl]piperazine-N′-[2-ethanesulfonic acid] (HEPES).

The polyethylene glycol diacrylate typically has a molecular weight between about 700 and about 800 and may be provided in a proportion ranging from about 50 to about 55 weight percent. The pentaerthyritol tetra 3(mercaptopropionate) may be provided in a proportion ranging from about 0.31 to about 0.53 times weight percent of the polyethylene glycol diacrylate present. If desired saline or other inert biocompatible materials may be added to the three component embolic material.

Optionally, the method may comprise temporarily reducing a blood flow through the endovascular graft and delivering an embolic material into the perigraft space while the blood flow through the endovascular graft is reduced or halted. The blood flow is substantially stopped through the endovascular graft and/or the perigraft space during the delivery of the embolic material so as to reduce, and preferably stop, the amount of distal perfusion of the embolic material from the perigraft space. The temporarily quiescent blood residing in the perigraft space allows for the injection of the embolic material into the perigraft space without concern for excessive distal flow of the embolic material out of the aneurysm sac. The blood flow may be reduced by positioning an occlusion member in the artery upstream of the endovascular graft. The occlusion member may take many forms but is typically in the form of an expandable balloon. The blood flow through the endovascular graft may be restored after the embolic material has substantially cured by deflating the expandable balloon.

Access to the perigraft space for injection of the embolic material may be achieved endoluminally or percutaneously translumbar. For example, the embolic material may be endovascularly injected into the perigraft space with a catheter which has its distal tip positioned between the endovascular graft and the artery wall. Additionally or alternatively, the embolic material may be percutaneously injected into the perigraft space with a delivery device, such as a syringe and a translumbar needle.

Upon delivery of the embolic material into the perigraft space, the embolic material may be in contact with an outer surface of the endovascular graft and an inner surface of the compromised portion of the artery wall. The embolic material may be radiopaque such that the radiopaque embolic material may be fluoroscopically monitored during the delivery of the radiopaque embolic material into the perigraft space. The embolic material typically has a first viscosity upon delivery into the perigraft space and a progressively higher viscosity as the material begins to cure. After the embolic material has substantially cured, it typically becomes a solid. The embolic material may exhibit, for example, a cure time between about approximately one minute and approximately ten minutes.

Various chemistries, cure times, viscosities, and radiopacities may be employed for the embolic material to facilitate the procedure and to allow optimum leak sealing while keeping the aortic occlusion time low. Cure times of the embolic material may be varied, as can the amount of dwell time of the embolic material prior to injecting the embolic material into the perigraft space so as to achieve a desired working time, while keeping the aortic occlusion times low.

If desired, the site of the endoleak and/or a flow pattern of the embolic fluid may first be identified before delivering the embolic material into the perigraft space. Typically, while the aortic flow is occluded by the occluding member, a contrast fluid may be injected into the perigraft space (e.g., aneurysm sac) to confirm the position of the endoleak and/or a distribution path of the contrast fluid material in the perigraft space using fluoroscopy or a like technique.

In some methods, the endovascular graft may be deployed in the artery just prior to the delivery of the embolic material into the perigraft space. At least a portion of the endovascular graft may be inflated with an inflation material. The inflation material may be used to inflate at least one of an inflatable cuff and an inflatable channel on the endovascular graft. The inflatable cuff may include a proximal and a distal cuff. The inflation material may be the same composition as the embolic material or it may be a different composition as the embolic material. In such methods, delivery of the embolic material around the endovascular graft may prevent the formation of endoleaks and would not require a separate surgical procedure to deliver the embolic material.

In another aspect, embodiments of the present invention provide systems for delivering an embolic material into a perigraft space. The systems may include a delivery device configured to access the perigraft space and configured to deliver an embolic material to the perigraft space. An occlusion assembly is configured to substantially reduce a blood flow through the endovascular graft during delivery of the embolic material. The embolic material may comprise polyethylene glycol diacrylate, pentaerthyritol tetra 3(mercaptopropionate), and a buffer.

The delivery device can be in a variety of forms. For example, the delivery device may comprise a syringe or a catheter. The occlusion assembly may include an occlusion member positioned adjacent a distal end of a guidewire. The occlusion member may be an expandable balloon.

The embolic material may be radiopaque. The buffer may be HEPES or glycylglycine. The glycylglycine may be provided in a proportion ranging from about 5 to about 40 weight percent. The polyethylene glycol diacrylate may have a molecular weight between 700 and 800 and may be provided in a proportion ranging from about 50 to about 55 weight percent. The pentaerthyritol tetra 3(mercaptopropionate) may be in a proportion ranging from about 0.31 to about 0.53 times the weight percent of the polyethylene glycol diacrylate present.

The embolic material may further comprise saline or other inert biocompatible materials. The saline may be in a proportion ranging between about 20 to about 50 percent by volume.

In a further aspect, the present invention provides a kit for depositing an embolic material in a perigraft space between an endovascular graft and an artery wall. The kit may comprise a delivery device configured to access the perigraft space and an embolic material comprising polyethylene glycol diacrylate, pentaerthyritol tetra 3(mercaptopropionate), and a buffer.

The delivery device may be a catheter configured to endovascularly access the perigraft space or a syringe that is configured to percutaneously access the perigraft space.

The buffer may comprise a glycylglycine buffer, and may be present in a proportion ranging from about 5 to about 40 weight percent. The polyethylene glycol diacrylate typically comprises a molecular weight between 700 and 800 and may be present in a proportion ranging from about 50 to about 55 weight percent. The pentaerthyritol tetra 3(mercaptopropionate) may be present in a proportion ranging from about 0.31 to about 0.53 times the weight percent of the polyethylene glycol diacrylate present.

The kits may further include instructions for use setting forth any of the methods described herein. Optionally, the kits may include an occlusion assembly for reducing the flow of blood through the deployed endovascular graft during the embolization procedure. The occlusion assembly may include an occlusion member that is in the form of an inflatable balloon.

The kits may also include packaging suitable for containing the delivery device, embolic material, and the instructions for use. Exemplary containers include pouches, trays, boxes, tubes, and the like. The instructions for use may be provided on a separate sheet of paper or other medium. Optionally, the instructions may be printed in whole or in part on the packaging. Usually, at least the delivery device and the occlusion assembly will be provided in a sterilized condition. Other kit components, such as a guidewire or an endovascular graft, may also be included.

These and other aspects of the invention will become more apparent from the following detailed description of the invention when taken in conjunction with the accompanying exemplary drawings.

### BRIEF DESCRIPTION OF THE DRAWINGS

FIG. 1 schematically illustrates a bifurcated endovascular graft positioned in an abdominal aortic aneurysm.

FIG. 2 schematically illustrates a temporary reduction of blood flow through the endovascular graft of FIG. 1.

FIG. 3 schematically illustrates delivery of a contrast fluid or dye into the perigraft space.

FIG. 4 illustrates a cured embolic material in the perigraft space.

FIG. 5 illustrates a system according to an embodiment of the present invention.

FIG. 6 illustrates a kit according to an embodiment of the present invention.

FIGS. 7 through 9 illustrate various endovascular grafts according to embodiments of the present invention.

FIGS. 10 through 12 illustrate various endovascular grafts according to alternative embodiment of the present invention.

### DETAILED DESCRIPTION OF THE INVENTION

The present invention provides methods and compositions for sealing endoleaks in a perigraft space between an endovascular device and a wall of a body lumen, such as an artery. For ease of discussion, the remainder of the discussion focuses on managing endoleaks associated with endovascular treatment of an abdominal aortic aneurysm (AAA) in which the body lumen is an artery; namely, the aorta. It should be appreciated however, that the embodiments of the present invention may also be used for the treatment of disease or injury that potentially compromises the integrity of other arteries and other flow conduits or lumens in the body. For example, embodiments of the present invention may be useful in treating indications in the digestive and reproductive systems as well as other indications in the cardiovascular system, including thoracic aortic aneurysms, arterial dissections (such as those caused by traumatic injury), etc.

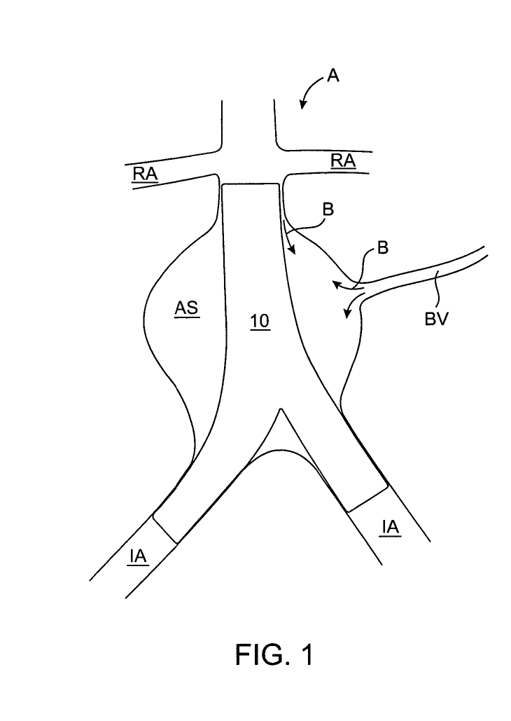

FIG. 1 schematically illustrates a bifurcated endovascular graft 10 deployed in a diseased aorta. Unless otherwise stated, the term “graft” or “endovascular graft” is used herein to broadly refer to a prosthesis capable of repairing and/or replacing diseased vessels or portions thereof, including generally tubular and bifurcated devices and any components attached or integral thereto.

For the purposes of this application, with reference to endovascular graft devices, the term “proximal” describes the end or portion of the graft that will be oriented towards the oncoming flow of bodily fluid, typically blood, when the device is deployed within a body passageway. The term “distal” therefore describes the graft end or portion opposite the proximal end.

The term “perigraft space” is used herein to define the space between an outside surface of the endovascular graft and the inside surface of a body lumen (e.g., an artery such as the aorta), typically including the aneurysm sac, from the proximal end of the graft to the distal end or ends of the graft.

Finally, while the drawings in the various figures are accurate representations of the various embodiments of the present invention, the proportions of the various components thereof are not necessarily shown to exact scale within, among, or between any given figure(s).

As shown in FIG. 1, endovascular graft 10 may be positioned to exclude an aneurysm sac AS or an otherwise diseased portion of the aorta from blood flow. As illustrated, aneurysm sac AS typically is proximal to the iliac arteries IA and distal of the renal arteries RA. In the illustrated embodiment, endovascular graft 10 is positioned in an infrarenal configuration, in which the endovascular graft is deployed below or distal to the renal arteries RA. In other embodiments, however, endovascular graft 10 may be positioned in a suprarenal configuration, such that the endovascular graft is fixed to the aorta proximal to the renal arteries (not shown). This would be the case, for instance, with a fenestrated graft that provided holes or fenestrations in the graft body to allow perfuision of the renal arteries RA.

Endovascular graft 10 is designed to exclude the aneurysm sac AS from blood pressure by redirecting blood flow through its central lumen. But in some instances, due to device migration or an aneurysm morphology change, for instance, blood B may still flow into aneurysm sac AS via incomplete sealing at the proximal or distal ends (i.e., a Type I endoleak), or via branch vessels BV, such as an interior mesenteric artery (IMA), lumbar arteries, etc. (i.e., a Type II endoleak).

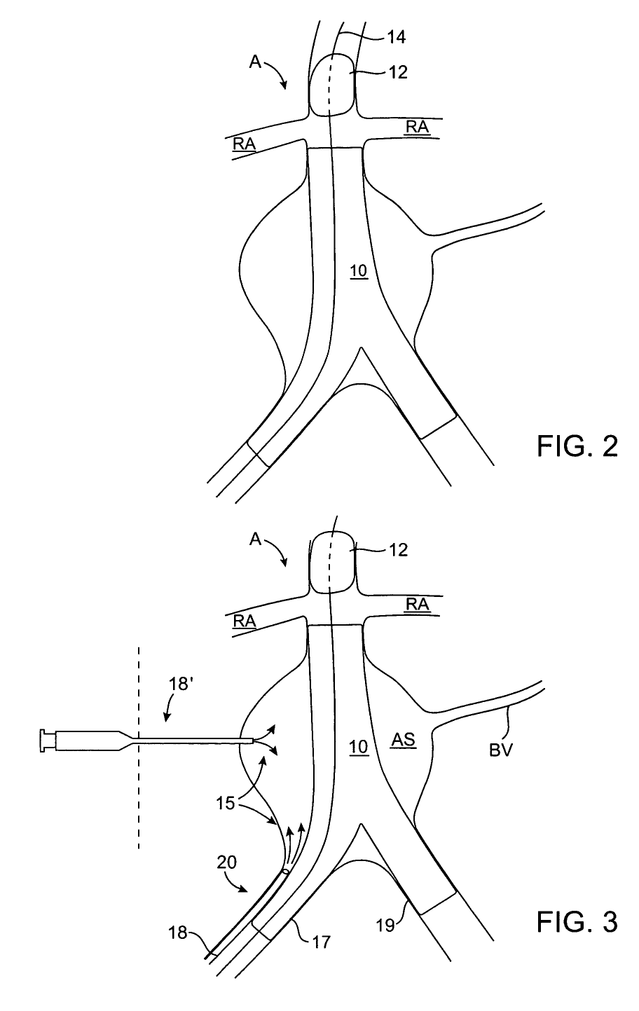

FIGS. 2 to 4 illustrate a method of managing endoleaks in the perigraft space according to an embodiment encompassed by the present invention. An occlusion member 12 may be advanced through the vasculature in a constrained configuration (not shown) to a position that is proximal to endovascular graft 10. Access to the vasculature may be achieved via the femoral artery and advancement of occlusion member 12 through the vasculature may be carried out using conventional catheter or guidewire-based delivery methods. The position of occlusion member 12 may be tracked under fluoroscopy as the occlusion member is advanced to the desired location. For example, all or a portion of the occlusion member and/or guidewire may be radiopaque. Once the occlusion member 12 has been advanced to the desired location, the occlusion member may be actuated to temporarily reduce, and typically substantially stop, the flow of blood from the aorta into endovascular graft 10 and aneurysm sac AS.

As illustrated in FIG. 2, occlusion member 12 is positioned proximal to the major branch vessels (e.g., renal arteries, celiac arteries, superior mesenteric arteries (SMA), etc. and are generically referred to in FIG. 2 as RA) to temporarily reduce and preferably stop the blood flow into aneurysm sac AS and endovascular graft 10 via the aorta A. It is generally desirable that occlusion member 12 be positioned proximal to the superior mesenteric arteries SMA (not shown) to prevent perfusion of the aneurysm sac AS via the inferior mesenteric arteries IMA (not shown) via systemic blood flow. As may be appreciated however, occlusion member 12 may be positioned distal of one or more of the major branch vessels, if desired. Such distal positioning may be desirable in the case, for instance, in which an inferior mesenteric artery IMA is thrombosed and the endoleak originates elsewhere.

Occlusion member 12 may be in the form of an expandable aortic balloon that is positioned at or near a distal end of a guidewire 14. The aortic occlusion balloon may be delivered through the artery on guidewire 14 in a constrained configuration (not shown). Once balloon 12 is positioned in the desired location in the aorta, balloon 12 may be expanded to an expanded configuration by delivery of an optionally radiopaque inflation fluid through an inflation lumen (not shown). Deflation of balloon 12 may be carried out by removing the inflation fluid from the balloon. The inflation lumen may be coupled to guidewire 14 or may be an inner lumen of a hollow catheter.

As shown in FIG. 3, after the occlusion member 12 is positioned in the aorta to create temporarily quiescent blood, a “forerunner” contrast fluid 15 may optionally be injected into the perigraft space via one or more delivery devices 18, 18′ so that the physician may readily view and confirm a path and distribution pattern of the embolic fluid that will be introduced into the perigraft space while the blood flow through endovascular graft 10 is stopped. Optionally, delivery devices 18, 18′ or other aspiration devices (not shown) may be used to aspirate aneurysm sac AS prior to delivery of the contrast fluid. As can be appreciated, such aspiration, however, is often unnecessary unless the endoleak is very small since introduction of the embolic material may displace fluid that is present in aneurysm sac.

Deflation of the aortic occlusion balloon 12 allows the contrast fluid to dissipate from the aneurysm sac by resumed blood flow through the perigraft space over a period of time. Dissipation of contrast fluid 15 allows the user to later see that the embolic material is adequately distributed within the aneurysm sac AS.

Once the contrast fluid has substantially dissipated from the perigraft space, the aortic occlusion balloon 12 may be reinflated to reduce, and typically substantially stop, the flow of blood into the endovascular graft (and possibly the perigraft space). The halted or otherwise reduced flow into the endovascular graft and/or perigraft space allows for the injection and curing of the embolic material in the perigraft space without the concern of excessive distal flow of the embolic material.

The perigraft space may be accessed using a variety of delivery devices to deposit the contrast fluid into the aneurysm sac. For example, as shown in FIG. 3, access to the perigraft space may be achieved endoluminally with a single lumen or multi-lumen catheter 18. A distal end 20 of catheter 18 may be guided into a space between the endovascular graft 10 and the arterial wall during or after deployment of the endovascular graft. Catheter 18 may be directed between the iliac artery and the ipsilateral leg 17 of the graft, the contralateral leg 19 of the graft, or both. While not shown, it may be possible to access the perigraft space proximally through the aorta or through the branch vessels BV, if desired. Access to the perigraft space via branch vessels BV, when they are patent, is generally desirable as such access minimizes the potential for disruption of the endovascular graft 10 seal due to passage of catheter 18 between the graft 10 and the arterial wall.

Alternatively or additionally, the aneurysm sac may be accessed directly translumbar with one or more delivery devices 18′, such as a syringe and an appropriate needle, so as to percutaneously deliver the contrast fluid directly into the perigraft space. As may be appreciated, syringe 18′ or another syringe (not shown) may also be used to aspirate any blood or other material from the perigraft space.

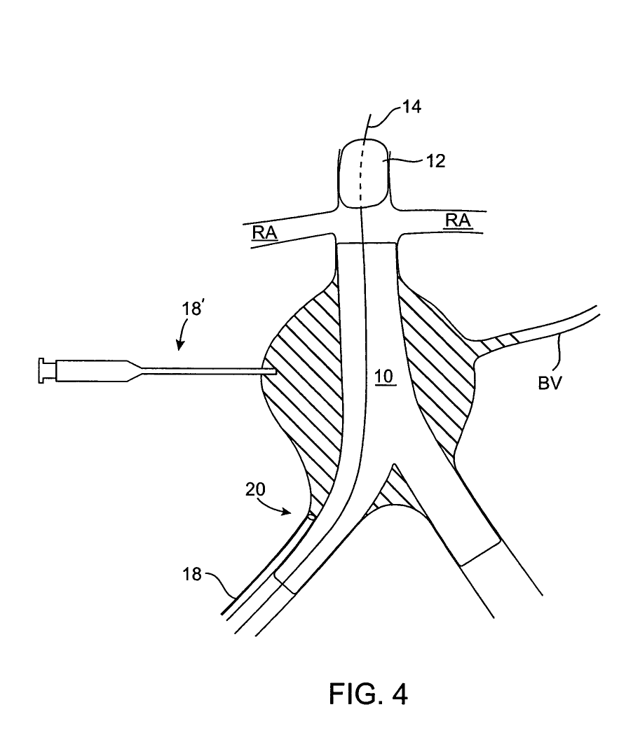

As shown in FIG. 4, the single lumen or multi-lumen catheter 18 and/or syringe 18′ may be used to deliver the multiple-component embolic material of the present invention into the perigraft space so that the embolic material contacts an outer surface of the endovascular graft 10 and a surface of the compromised portion of the aortic wall (e.g., aneurysm sac wall) so as to treat the endoleak(s). Once the embolic material has substantially cured, as discussed below, occlusion member 12 may be deflated and the blood flow through the endovascular graft may be restored.

One example of a suitable catheter 18 is an angiographic catheter with a radiopaque tip. Such a catheter would provide an adequate flow lumen (to allow manual injection of embolic material with a syringe) and facilitate location of the catheter end at the appropriate site within the aneurysm. Such a catheter could have an outer diameter up to about 0.035″ or about 0.038″, and be guidewire compatible, and are readily available in operating rooms, catheterization labs, or radiology suites where endovascular interventions are routinely performed. As can be appreciated, however, the present invention is not limited to angiographic catheters and many other types of conventional and proprietary catheters may be used to deliver the embolic material.

As may be appreciated, in some embodiments it may be desirable to use separate catheters or syringes (not shown) to deliver the contrast fluid and embolic material to the perigraft space. Alternatively, heparanized saline flush may be used to clear contrast fluid from a single-lumen catheter 18 prior to the introduction of the embolic material through catheter 18.

For embolic materials with a longer cure time, the embolic material may be injected into the perigraft space in a less precise or specific locations, and the embolic material may be allowed to flow to the Type I endoleaks on the proximal or distal ends of the endovascular graft and/or penetrate into the branch vessels (e.g., for sealing of Type II endoleaks), so as to embolize and close off the leak paths. Depending on the characteristics of the embolic material, if a blood flow through the perigraft space and endovascular graft is not stopped or substantially reduced, the embolic material may perfuse from the perigraft space prior to curing and sealing of the endoleaks and may create potential embolic complications in the bowels or peripheral circulation.

As may be appreciated, while some embodiments of the present invention reduce, and typically substantially stop the flow of blood through the endovascular graft and/or aneurysm sac prior to the sealing of the endoleaks, the viscosity and curing time of the embolic material may be chosen such that the occlusion member 12 is not needed during the procedure.

Useful embolic materials generally include those formed by the mixing of multiple components and that have a cure time ranging from a few minutes or less to tens of minutes, preferably from about one to about ten minutes such that the embolic material is allowed to penetrate into the targeted branch vessels and/or penetrate into the endoleak, but not beyond. Depending on the composition, the embolic material may be mixed in vivo or in vitro. Such a material should be biocompatible, exhibit long-term stability (preferably but not necessarily on the order of at least ten years in vivo), and exhibit adequate mechanical properties, both pre- and post-cure, suitable for service in the aneurysm sac of the present invention in vivo. For instance, such a material should have a relatively low viscosity before solidification or curing to facilitate the process of filling the desired volume. The embolic material may be radiopaque, both acutely and chronically, although this is not necessary.

One class of suitable materials for embolization is the family of Michael addition polymers formed by reaction of an acrylate monomer and a multi-thiol. These materials can be delivered in liquid or semi-liquid form, and thereafter crosslink in situ to form a solid polymer gel. Details of the Michael addition polymer class of compositions suitable for use as an embolic material are described in U.S. patent application Ser. No. 09/496,231 to Hubbell et al., filed Feb. 1, 2000 and entitled “Biomaterials Formed by Nucleophilic Addition Reaction to Conjugated Unsaturated Groups” and U.S. patent application Ser. No. 09/586,937 to Hubbell et al., filed Jun. 2, 2000 and entitled “Conjugate Addition Reactions for the Controlled Delivery of Pharmaceutically Active Compounds”. The entirety of each of these patent applications are hereby incorporated herein by reference.

One Michael addition material suitable for endoleak management applications is a polymer formed by mixing polyethylene glycol diacrylate (PEGDA) with pentaerythrithritol tetra (3-mercaptopropionate) (QT). A buffer such as glycylglycine or other suitable compound may be added to adjust the solidification time and/or the viscosity of the liquid components prior to curing as described below in greater detail.

A radiopaque agent may also be added to facilitate visualization of the embolization material under fluoroscopy and/or on follow-up imaging modalities such as computed tomography (CT). Suitable radiopaque agents include relatively insoluble materials such as barium sulfate and tantalum, and soluble materials such as iodinated contrast agents. Tantalum is a particularly useful agent in this regard as it reduces the potential for late dissipation of radiopacity due to its low solubility compared to barium sulfate and its potential for promoting thrombosis.

In general, we have found that the PEGDA/QT ratio may vary for a given PEGDA molecular weight, but preferably this ratio should vary in a defined range. For instance, for a PEGDA molecular weight of 742, we have found that PEGDA present in a proportion ranging from about 1.9 to about 3.2 times the amount of QT present, by weight, is useful. Another useful formulation of this PEGDA/QT/buffer material may comprise:
(1) PEGDA having a molecular weight of between about 700 and 800; preferably between about 740 and 760; more preferably about 750, present in a proportion ranging from about 50 to about 55 weight percent; specifically in an overall proportion of about 53 weight percent,
(2) QT, present in a proportion ranging from about 0.31 to about 0.53 times the weight percent of the PEGDA present; specifically in an overall proportion of about 22 weight percent, and
(3) glycylglycine buffer, having a concentration of between about 100 millimole and about 500 millimole; preferably about 400 millimole, present in a proportion ranging from about 5 to about 40 weight percent; specifically in an overall proportion of about 25 weight percent.

Variations of these components and other formulations as described in copending U.S. patent application Ser. Nos. 09/496,231 and 09/586,937, both to Hubbell et al., may be used as appropriate. The entirety of each of these patent applications are hereby incorporated herein by reference. In addition, PEGDA having a molecular weight ranging from about 350 to about 850 may be useful; PEGDA having a molecular weight ranging from about 440 to about 750 are also particularly useful.

Other biological buffers, such as N-[2-hydroxyethyl]piperazine-N′-[2-ethanesulfonic acid] (HEPES), may be used instead of glycylglycine.

The strength of the buffer (as measured by its molarity) controls the pH of this embolic material, which in turn exclusively governs the material's cure time. Moreover, as the buffer typically is the least viscous of the three components described above, the volume of buffer present most efficiently affects the viscosity of the material before it cures. The influence of the buffer on the embolic material viscosity and cure time may be therefore be effected by controlling the buffer quantity and strength. We have found that when using glycylglycine in quantities ranging from between about 5 and about 40 weight percent as described above, and preferably about 25 weight percent, a concentration of approximately 400 millimole achieves a useful balance between the desired cure time and pre-cure viscosity.

It is within the scope of the present invention to adjust the strength and quantity of buffer in this three-component material to achieve the desired combination of properties (such as viscosity and cure time) for a given indication and delivery system. For instance, when managing endoleaks as described herein, it is generally desirable to increase the viscosity of the uncured material and thereby facilitate controlled placement of the material in vivo without the unintended perfusion of peripheral or secondary vascular beds. Viscosity may be increased for this and other embolic materials described herein by decreasing the buffer volume and increasing the buffer molarity. Bulking or thixotropic agents such as silica gel may be additionally or alternatively added in any combination as well.

A polymer formed by mixing ethoxylated trimethylolpropane triacrylate (ETMPTA) with QT may also be used as an effective embolic material. A buffer and/or a radiopaque agent may be used with this system. Another specific example material that may be used in the present invention is a polymer formed by mixing polypropylene oxide diacrylate (PPODA) with QT. A buffer and/or a radiopaque agent may also be used with this system.

An alternative to these three-component systems is a gel made via polymer precipitation from biocompatible solvents. Examples of such suitable polymers include ethylene vinyl alcohol and cellulose acetate. Examples of such suitable biocompatible solvents include dimethylsulfoxide (DMSO), n-methyl pyrrolidone (NMP) and others. Such polymers and solvents may be used in various combinations as appropriate. Other materials such as cyanoacrylates (such as TRUFILL from Cordis Corporation, Miami Lakes, Fla.) may be used as well.

Alternatively, various siloxanes may be used as an embolic material. Examples include hydrophilic siloxanes and polyvinyl siloxanes (such as STAR-VPS from Danville Materials of San Ramon, Calif. and various silicone products such as those manufactured by NuSil, Inc. of Santa Barbara, Calif.).

Other gel systems useful as an embolic material for the embodiments of the present invention include phase change systems that gel upon heating or cooling from their initial liquid or thixotropic state. For example, materials such as n-isopropyl-polyacrylimide (NIPAM) are suitable.

Effective gels may also comprise thixotropic materials that undergo sufficient shear-thinning so that they may be readily injected through a conduit such as a delivery catheter or syringe but yet still are able to become substantially gel-like at zero or low shear rates.

Cure times may be tailored by adjusting the formulations, mixing protocol, and other variables according to the requirements of the clinical setting.

In the various embodiments of the present invention, it is desirable that the embolic material be visible through the use of techniques such as fluoroscopy during the time of delivery in which the perigraft space is being filled with the embolic material. Such visibility allows the clinician to monitor and verify that the aneurysm sac, endoleaks, and/or branch vessels are filling correctly and to adjust the delivery procedure if they are not. It also provides an opportunity to detect any leakage or otherwise undesirable flow of the embolic material out of the perigraft space so that the injection may be stopped, thereby minimizing the amount of distal perfusion of the embolic material.

It is also desirable that the cured embolic material be visible through the use of follow-up imaging techniques such as computed tomography (CT) and the like.

While the above embolic materials are examples of preferred materials that may be used with the methods of the present invention, it may be appreciated that other conventional and proprietary embolic materials may be used with the methods of the present invention to seal the endoleaks.

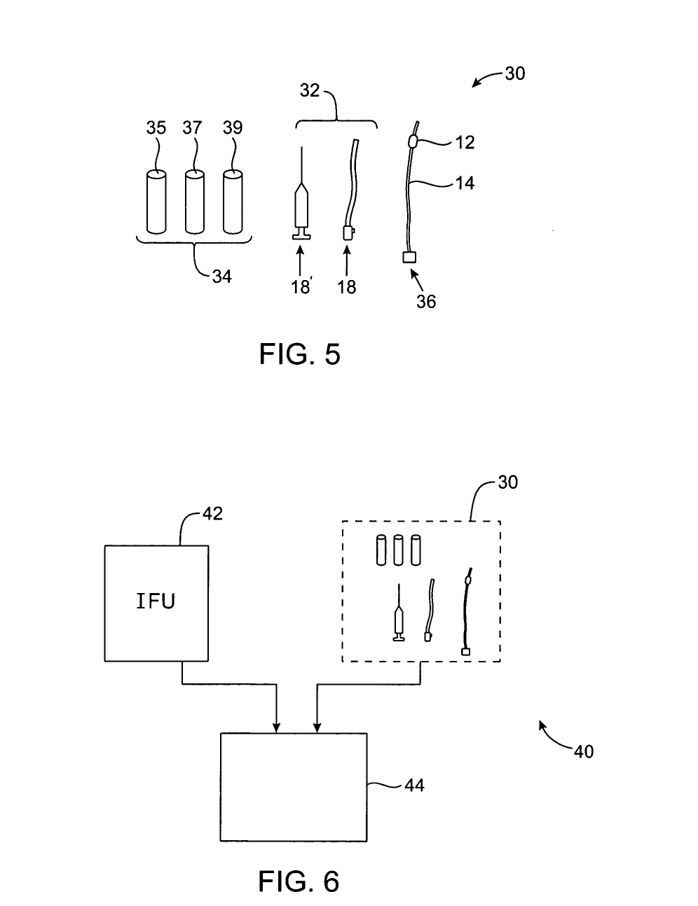

FIG. 5 illustrates a system 30 for managing endoleaks according to an embodiment of the present invention. System 30 includes a delivery device 32 for accessing the perigraft space. Delivery device 32 may include one or more of a catheter 18, a syringe and needle 18′, or other conventional devices that may be used to access a perigraft space. System 30 also includes an embolic material 34 that is deliverable by delivery device 18 into the perigraft space. The embolic material may be a three-component mixture, such as a mixture of polyethylene glycol diacrylate, pentaerthyritol tetra 3(mercaptopropionate), and a buffer. In the illustrated embodiment, each of the separate components of the embolic material are stored in separate containers 35, 37, 39 and are mixed together just prior to delivery. As can be appreciated, embolic material 34 may be composed of any of the other materials described herein.

System 30 may optionally include an occlusion assembly 36 that is configured to substantially reduce blood flow through a deployed endovascular graft and/or perigraft space. As described above in relation to FIGS. 2 to 4, one embodiment of occlusion assembly 36 is an inflatable occlusion member 12 coupled to a distal end of a catheter 14.

FIG. 6 illustrates one kit 40 according to an embodiment of the present invention. Kit 40 may include a combination of system 30, instructions for use 42, and one or more packages 44. Delivery device 32 will generally be as described above, and the instruction for use (IFU) 42 will set forth any of the methods described above. Package 44 may be any conventional medical device packaging, including pouches, trays, boxes, tubes, or the like. The instructions for use 42 will usually be printed on a separate piece of paper, but may also be printed in whole or in part on a portion of the package 44. Optionally, kit 40 may include a guidewire (not shown) for assisting in the positioning of the catheter 18, an endovascular graft 10, and/or a delivery system for delivering the endovascular graft (not shown).

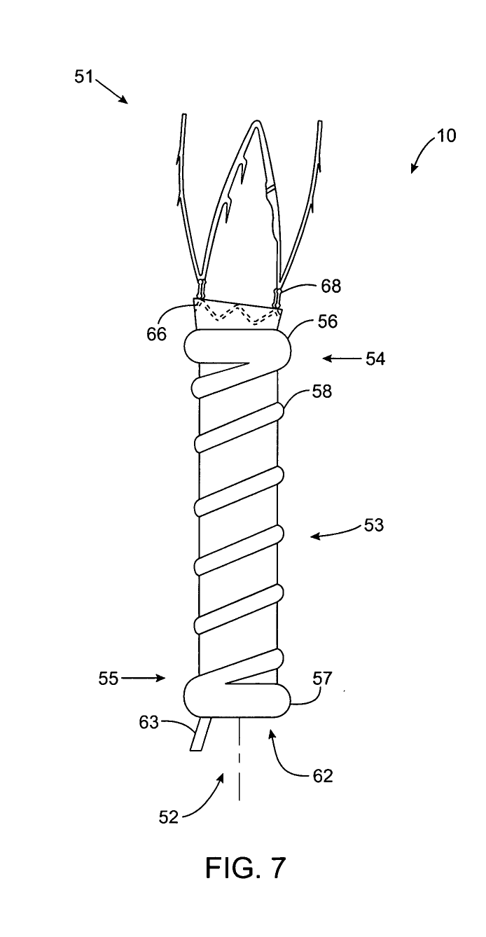

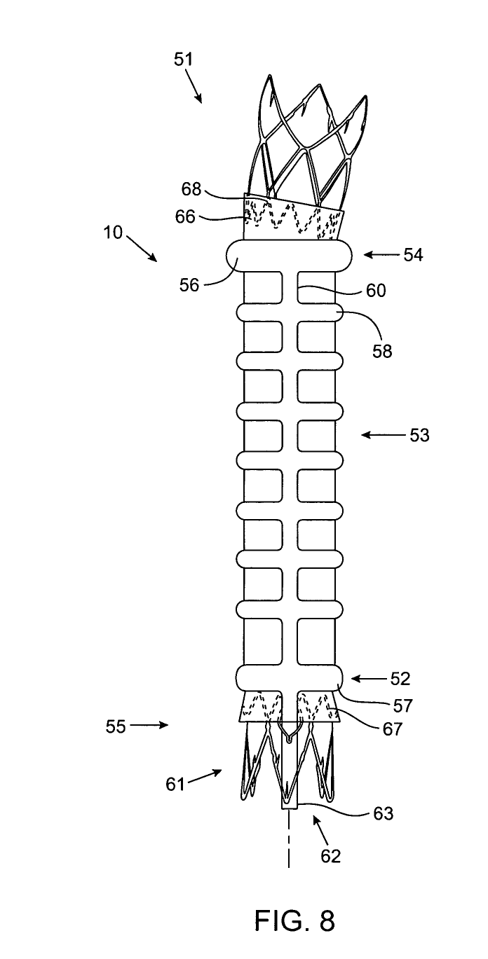

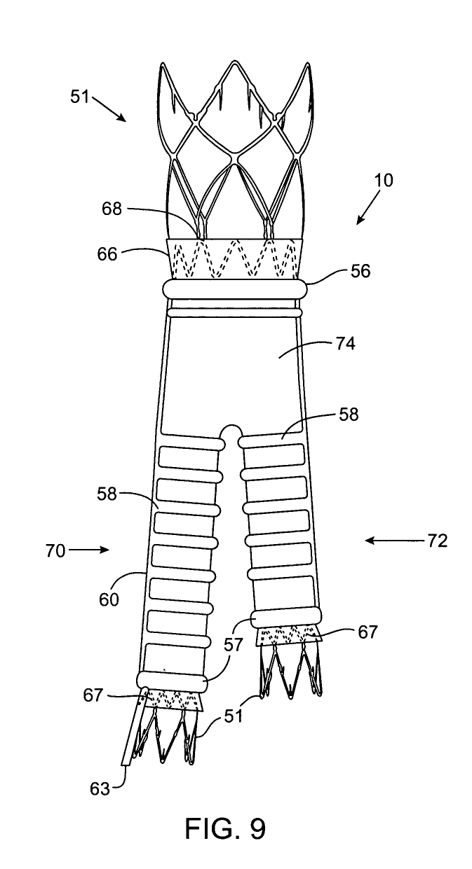

FIGS. 7 to 9 illustrate some examples of an endovascular graft 10 that may be used with the methods and systems of the present invention to isolate a diseased portion (e.g., aneurysm) of a body lumen, such as the aorta, from blood flow. The embodiments of FIGS. 7 and 8 are tubular, and the embodiment of FIG. 9 is bifurcated.

As shown in FIGS. 7 and 8, graft 10 has a proximal end 54 and a distal end 52 and includes a generally tubular structure or graft body section 53 comprised of one or more layers of fusible material, such as expanded polytetrafluoroethylene (ePTFE). A proximal inflatable cuff 56 is disposed at or near a proximal end 54 of graft body section 53 and an optional distal inflatable cuff 57 is disposed at or near a graft body section distal end 55. Graft body section 53 forms a longitudinal lumen 62 configured to confine a flow of fluid therethrough and may range in length from about 5 cm to about 30 cm; specifically from about 10 cm to about 20 cm.

A proximal connector member 66 may be embedded within multiple layers of graft body section 53 in the vicinity of graft body section proximal portion 54. In the embodiment of FIG. 7, the connector member is a serpentine ring. Other embodiments of connector member 66 may take different configurations. As shown in FIG. 8, a distal connector member 67 may also be embedded within multiple layers of graft body section 53 in the vicinity of graft body section distal portion 55.

One or more expandable members or stents 51, 61 may be coupled or affixed to either or both proximal connector member 66 and distal connector member 67 via one or more connector member connector elements 68. Such expandable members or stents may serve to anchor the endovascular graft 10 within the aorta and resist longitudinal or axial forces imposed on the endovascular graft 10 by the pressure and flow of fluids through the graft 10. In this embodiment, connector elements 68 of the proximal and distal connector members 66 and 67 extend longitudinally outside proximal end 52 and distal end 54 of endovascular graft 10, respectively.

FIG. 9 illustrates a bifurcated graft according to an embodiment of the present invention. A bifurcated device such as endovascular graft 10 may be utilized to repair a diseased lumen at or near a bifurcation within the vessel, such as, for example, in the case of an abdominal aortic aneurysm in which the aneurysm to be treated may extend into the anatomical bifurcation or even into one or both of the iliac arteries distal to the bifurcation. In the following discussion, the various features of the graft embodiments previously discussed may be used as necessary in the bifurcated graft 10 embodiment unless specifically mentioned otherwise.

Graft 10 comprises a first bifurcated portion 70, a second bifurcated portion 72 and main body portion 74. The size and angular orientation of the bifurcated portions 70 and 72, respectively, may vary—even between portion 70 and 72—to accommodate graft delivery system requirements and various clinical demands. For instance, each bifurcated portion or leg is shown in FIG. 9 to have a different length, but this is not necessary. First and second bifurcated portions 70 and 72 are generally configured to have an outer inflated diameter that is compatible with the inner diameter of a patient's iliac arteries. First and second bifurcated portions 70 and 72 may also be formed in a curved shape to better accommodate curved and even tortuous anatomies in some applications. A proximal inflatable cuff 56 is disposed at or near a proximal end 54 of main body section 74 and optional distal inflatable cuffs 57 may be disposed at or near one or both of the distal end of the first bifurcated portion 70 and the second bifurcated portion 72.

Similar to the embodiments of FIGS. 7 and 8, a proximal connector member 66 may be embedded within multiple layers of main body portion 74 and optionally, distal connector members 67 may be embedded within multiple layers of bifurcated portions 70, 72. One or more expandable members or stents 51 may be coupled or affixed to proximal connector member 66 and/or distal connector members 67 via one or more connector member connector elements 68.

As shown in FIGS. 7 to 9, and as will be described in greater detail below, inflation of cuffs 56, 57, in free space (i.e. when graft 10 is not disposed in a vessel or other body lumen) will cause them to assume a generally annular or torodial shape (especially when the graft body is in an unconstrained state) with a somewhat circular longitudinal cross-section. Inflatable cuffs 56, 57 will generally, however, conform to the shape of the vessel within which it is deployed. When fully inflated, cuffs 56, 57 may have an outside diameter ranging from about 10 mm to about 45 mm; specifically from about 16 mm to about 32 mm.

Referring now to FIG. 7, at least one inflatable channel 58 may be disposed between and in fluid communication with proximal inflatable cuff 56 and distal inflatable cuff 57. The inflatable channels 58 (and inflatable cuffs 56, 57) may be integrally formed in the body section 53 by seams formed in the body section 53. The network of inflatable cuffs 56, 57, and channel 58 may be inflated, most usefully in vivo, by introduction or injection of an inflation material or medium through an injection port 63 that is in fluid communication with cuff 57 and the associated cuff/channel network.

As shown in FIG. 8, some embodiments may include a longitudinal inflatable channel 60 that communicates with the inflatable channel 58 and inflatable cuffs 56, 57. Inflatable channel 58 provides structural support to graft body section 53 when inflated to contain an inflation medium. Inflatable channel 58 further prevents kinking and twisting of the tubular structure or graft body section when it is deployed within angled or tortuous anatomies as well as during remodeling of body passageways (such as the aorta and iliac arteries) within which graft 10 is deployed. Channels 58 may take on a variety of forms but are typically in a parallel, linear or helically configuration. Together with proximal and distal cuffs 56 and 57, inflatable channel 58 forms a network of inflatable cuffs and channels in fluid communication with one other.

Referring again to FIG. 9, first and second bifurcated portions 70 and 72 may also comprise a network of inflatable cuffs and channels, including inflatable channels. Channels comprise one or more optional inflatable longitudinal channels 60 (e.g., a spine) in fluid communication with one or more approximately parallel inflatable circumferential channels 58, all of which are in fluid communication with optional distal inflatable cuffs 57. Channels 58 may take on a variety of forms but are typically in a parallel, linear configuration. Channels 58 may take the form of a helix, for example, which would combine the functions of the parallel circumferential channels 58 and longitudinal channels 60.

In the embodiment of FIG. 9, channel 58 forms a continuous cuff and channel network extending from first bifurcated portion 70 to main body portion 74 to second bifurcated portion 72. Accordingly, inflatable channel 58 fluidly connects into a network with proximal inflatable cuff 56, optional distal inflatable cuffs 57. Note that spine or longitudinal channels 60 extend proximally along main body portion 74 to be in fluid communication with cuffs 56 and 57.

The network of inflatable cuffs 56, 57, and channel 58 may be inflated, most usefully in vivo, by introduction or injection of an inflation material or medium through an injection port 63 that is in fluid communication with cuff 57 and the associated cuff/channel network. The inflation material may comprise one or more of a solid, fluid (gas and/or liquid), gel or other medium. The inflation material may contain a contrast medium that facilitates imaging the device while it is being deployed within a patient's body. For example, radiopaque materials containing elements such as bismuth, barium, gold, iodine, platinum, tantalum or the like may be used in particulate, liquid, powder or other suitable form as part of the inflation medium. Liquid iodinated contrast agents are a particularly suitable material to facilitate such imaging. Radiopaque markers may also be disposed on or integrally formed into or on any portion of graft 10 for the same purpose, and may be made from any combination of biocompatible radiopaque materials.

In one embodiment, the inflation material is the same material that is used as the embolic material, such as those described herein. In other embodiments, the inflation material may be a different material than the embolic material. In such embodiments, the inflation material and embolic material may be configured to provide the mechanical characteristics that are desirable for their specific purpose. For example, in the proximal and distal cuffs 56, 57 of the various embodiments of the present invention, the inflation material serves as a conformable sealing medium to provide a seal against the lumen wall. Desirable mechanical characteristics for the inflation medium in the proximal and distal cuffs would therefore include a low shear strength so to enable the cuffs 56, 57 to deform around any luminal irregularities (such as calcified plaque asperities) and to conform to the luminal profile, as well as a high volumetric compressibility to allow the embolic material to expand the cuffs as needed to accommodate any late lumen dilatation and maintain a seal.

In the channel or channels 58, 60 by contrast, the inflation medium serves primarily to provide structural support to the lumen within which the graft is placed and kink resistance to the graft. Desirable mechanical characteristics for the inflation medium in the channel or channels therefore includes a high shear strength, to prevent inelastic deformation of a channel or channel segment due to external compression forces from the vessel or lumen (due, for example, to neointimal hyperproliferation) and low volumetric compressibility to provide stable support for adjacent channels or channel segments that may be in compressive contact with each other, thereby providing kink resistance to the graft.

Finally, in the perigraft space, it is desired that the embolic material cure time be controlled, typically by ensuring it cures relatively quickly (from times ranging from about one minute or less to tens of minutes) after introduction into the perigraft space, so as to reduce the possibility that the embolic material migrates into undesirable portions of the vasculature. Desirable mechanical characteristics for the embolic material in the perigraft space include high volumetric and chemical stability, given that the embolic material typically is in direct contact with either or both tissue and blood.

Given these contrasting requirements, it may be desirable to have different inflation materials fill different portions of the graft, such as one inflation medium for the proximal and distal cuffs and a second in the channel or channels and a different embolic material to manage the endoleaks.

In some methods of the present invention, it may be desirable to fill the perigraft space before the endoleaks are even formed. In such embodiments, the embolic material may be delivered into the perigraft space immediately after the endovascular graft 10 is deployed in the AAA or other diseased portion of the aorta. Such methods generally follow similar method steps described above.

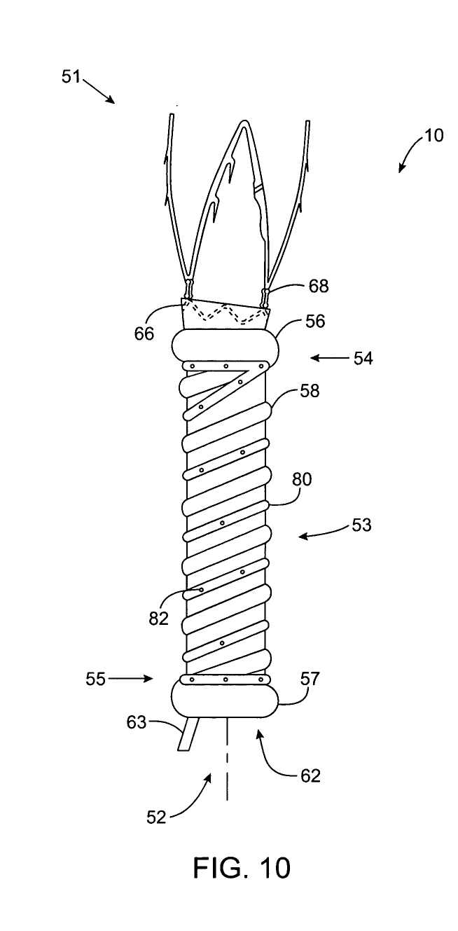

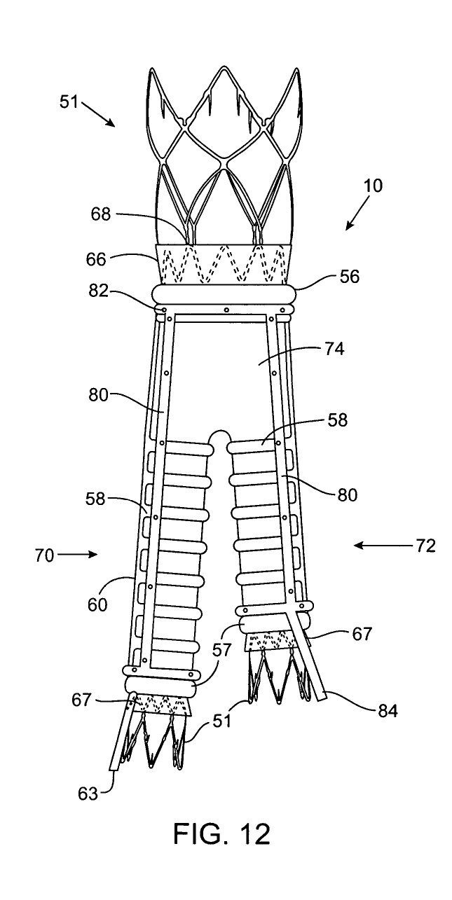

Some alternative configurations of grafts suitable for the present invention are illustrated schematically in FIGS. 10-12. The alternative configurations comprise an inflatable graft, such as the ones described and referred to herein in conjunction with FIGS. 7-9. In the embodiments of FIGS. 10-12, a separate lumen, channel, or network of lumens or channels 80 may be incorporated into the graft to deliver the embolic material to the perigraft space.

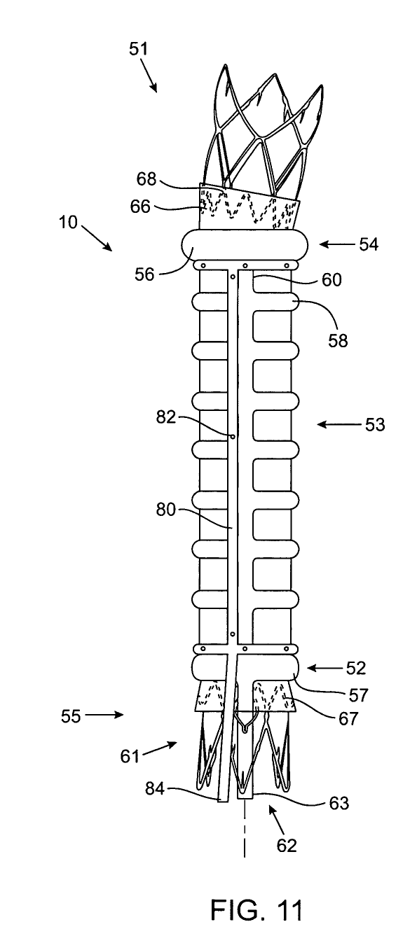

The embolic material may be delivered into the perigraft space via the embolic material delivery channels or lumen 80 in a variety of ways. For instance, the embolic material may be delivered to channels 80 via an injection port 84 (which may be similar to (FIG. 11) or the same as (FIG. 10) injection port 63). The embolic material may travel through channel 80 and exit channel 80 into the perigraft space through one or more abluminal apertures or openings 82 in the channels. Some useful aperture configuration are shown in FIGS. 10-12. The examples show that the one or more apertures 82 are disposed (1) near the proximal cuff 56 of the graft, (2) in the mid-graft region (and preferably configured to be oriented towards the aneurysm sac AS upon deployment to facilitate filling of the perigraft space), and/or (3) in a region of the graft near the distal cuff 57.

If desired, apertures 82 may be longitudinally symmetrically distributed over the graft to ensure that all parts of the perigraft space is filled at a substantially equal rate. In other configurations, apertures 82 may be positioned asymmetrically over the graft. Alternatively or in addition to the above, one or more embolic material delivery channels may have an open distal end or terminus through which the embolic material may enter the perigraft space. It should be appreciated, however, that any number of apertures may be used as needed in a variety of locations and configurations, and the present invention is not limited to the illustrated examples of FIGS. 10-12.

Channels 80 may be the same size, larger or smaller than inflatable lumen channels 58. Channels 80 may be positioned anywhere on the graft body, but typically overlap inflatable lumen channels and/or are interspersed between inflatable lumen channels 58. Aperture(s) 82 may have any shape and size, but are typically round and have a diameter between about 0.5 mil and about 2.0 mils.

Delivery of embolic material in conjunction with the various inflatable grafts described herein may take place prior to, simultaneous with, or after inflation of the network of cuffs and channels in the graft. Desirably, the embolic material is delivered after the graft is filled so to aid in controlling distal perfusion.

Various embodiments of grafts and stent-grafts, methods of manufacturing the grafts, and methods of delivering the grafts are described in co-pending and commonly owned U.S. patent application Ser. No. 10/029,557, entitled “Method and Apparatus for Manufacturing an Endovascular Graft Section”, U.S. patent application Ser. No. 10/029,570, entitled “Method and Apparatus for Shape Forming Endovascular Graft Material”, U.S. patent application Ser. No. 10/029,584, entitled “Endovascular Graft Joint and Method of Manufacture“, by Chobotov et al., all of which were filed Dec. 20, 2001, U.S. patent application Ser. No. 10/327,711, entitled “Advanced Endovascular Graft”, by Chobotov et al., filed Dec. 20, 2002, PCT Application No. PCT/US02/40997, entitled “Method and Apparatus for Manufacturing an Endovascular Graft,” by Chobotov et al., filed Dec. 20, 2002, U.S. patent application Ser. No. 09/774,733, entitled “Delivery System and Method for Expandable Intracorporeal Device,” by Chobotov et al, filed Jan. 31, 2002 and U.S. patent application Ser. No. 10/122,474, entitled “Delivery System and Method for Bifurcated Endovascular Graft,” by Chobotov et al., filed Apr. 11, 2002, the entirety of each of which are incorporated herein by reference. Other embodiments of devices incorporating features and methods described herein are disclosed in U.S. Pat. No. 6,395,019 (May 28, 2002) to Chobotov, the entirety of which is incorporated herein by reference.

As may be appreciated, a variety of endovascular grafts may be used with the methods and embolic materials of the present invention, and the present invention is not limited to use with the endovascular stent-grafts described herein. For example, the embodiments of the present invention may be used with a stent, tubular graft, bifurcated graft, coated stent, covered stent, other configurations of unitary or modular stent-grafts, and the like, such as those sold by Medtronic, Inc. (Minneapolis, Minn.), W.L. Gore & Associates, Inc. (Newark, Del.), Cook Group, Inc. (Bloomington, Ind.), etc.

While particular forms of the invention have been illustrated and described, it will be apparent that various modifications can be made without departing from the spirit and scope of the invention.

1. A method of reducing a blood flow into a perigraft space between an endovascular graft and a body lumen wall, the method comprising:
accessing the perigraft space with a delivery device; and
delivering an embolic material into the perigraft space with the delivery device,
wherein the embolic material comprises polyethylene glycol diacrylate, pentaerthyritol tetra 3(mercaptopropionate), and a buffer.

2. The method of claim 1 comprising identifying a flow path of the embolic material within the perigraft space prior to delivering the embolic material into the perigraft space.

3. The method of claim 2 wherein identifying a flow path of the embolic material within the perigraft space comprises:
introducing a contrast fluid into the perigraft space; and
monitoring a flow pattern of the contrast fluid within the perigraft space.

4. The method of claim 1 comprising reducing a blood flow through the endovascular graft prior to delivery of the embolic material into the perigraft space.

5. The method of claim 4 further comprising:
introducing a contrast fluid into the perigraft space;
monitoring a flow pattern of the contrast fluid within the perigraft space; and
allowing the contrast fluid to substantially dissipate from the space between the endovascular graft and the body lumen wall by temporarily restoring blood flow through the endovascular graft.

6. The method of claim 4 wherein reducing the blood flow is carried out with an occlusion member that is positioned upstream of the endovascular graft.

7. The method of claim 6 wherein the occlusion member is an expandable balloon,
wherein reducing the blood flow through the endovascular graft comprises inflating the expandable balloon within the body lumen.

8. The method of claim 4 further comprising restoring the blood flow through the endovascular graft after the embolic material has substantially cured.

9. The method of claim 8 wherein the embolic material has a first viscosity upon delivery into the perigraft space and a solidifies after the embolic material has substantially cured.

10. The method of claim 4 wherein delivering the embolic material while the blood flow through the endovascular graft is reduced reduces the amount of distal perfusion of the embolic material from the perigraft space.

11. The method of claim 4 wherein reducing the blood flow comprises substantially stopping the blood flow through the endovascular graft and the perigraft space.

12. The method of claim 1 wherein the embolic material is radiopaque.

13. The method of claim 12 comprising fluoroscopically monitoring the delivery of the radiopaque embolic material into the perigraft space.

14. The method of claim 1 wherein the embolic material cures in situ to embolize the perigraft space, wherein the embolic material contacts an outer surface of the endovascular graft and an inner surface of the body lumen wall to reduce a blood flow into the perigraft space.

15. The method of claim 1 wherein the embolic material cures in approximately one minute to approximately ten minutes.

16. The method of claim 1 wherein the embolic material of the polyethylene glycol diacrylate, pentaerthyritol tetra 3(mercaptopropionate), and the buffer mixes in vitro.

17. The method of claim 1 wherein accessing the perigraft space comprises percutaneously positioning the delivery device in the perigraft space.

18. The method of claim 17 wherein delivering the embolic material comprises a translumbar injection of the embolic material into the perigraft space.

19. The method of claim 1 wherein the delivery device comprises a catheter with a distal tip, wherein accessing the perigraft space comprises endovascularly positioning the distal tip of the catheter between the endovascular graft and the body lumen wall.

20. The method of claim 1 wherein the buffer comprises glycylglycine.

21. The method of claim 20 comprising providing the glycylglycine buffer in a proportion ranging from about 5 to about 40 weight percent.

22. The method of claim 1 wherein the buffer comprises HEPES.

23. The method of claim 1 comprising providing the polyethylene glycol diacrylate in a proportion ranging from about 50 to about 55 weight percent.

24. The method of claim 1 wherein the polyethylene glycol diacrylate comprises a molecular weight between about 700 and about 800.

25. The method of claim 24 comprising providing the pentaerthyritol tetra 3(mercaptopropionate) in a proportion ranging from about 0.31 to about 0.53 times weight percent of the polyethylene glycol diacrylate present.

26. The method of claim 1 further comprising adding saline or other inert biocompatible materials to the embolic material.

27. The method of claim 1 further comprising:
deploying the endovascular graft in the body lumen prior to the delivery of the embolic material; and
inflating at least a portion of the endovascular graft with an inflation fluid.

28. The method of claim 27 wherein inflating at least a portion of the endovascular graft with the inflation fluid comprises filling at least one of an inflatable cuff and a fill channel with the inflation fluid.

29. The method of claim 28 wherein the embolic material and the inflation fluid are the same materials.

30. The method of claim 28 wherein the embolic material and the inflation fluid are different materials.

31. A system for depositing an embolic material in a perigraft space between an endovascular graft and a body lumen wall, the system comprising:
a delivery device configured to access the perigraft space;
an occlusion assembly that is configured to substantially reduce a blood flow through the endovascular graft; and
an embolic material that is delivered to the perigraft space with the delivery device, wherein the embolic material comprises polyethylene glycol diacrylate, pentaerthyritol tetra 3(mercaptopropionate), and a buffer.

32. The system of claim 31 wherein the occlusion assembly comprises an occlusion member positioned adjacent a distal end of a guidewire.

33. The system of claim 32 wherein the occlusion member is an expandable balloon.

34. The system of claim 31 wherein the delivery device comprises a syringe.

35. The system of claim 31 wherein the delivery device comprises a catheter.

36. The system of claim 31 wherein the embolic material is radiopaque.

37. The system of claim 37 wherein the buffer comprises glycylglycine.

38. The system of claim 37 wherein the glycylglycine buffer is in a proportion ranging from about 5 to about 40 weight percent.

39. The system of claim 37 wherein the buffer comprises HEPES.

40. The system of claim 37 wherein the polyethylene glycol diacrylate is in a proportion ranging from about 50 to about 55 weight percent.

41. The system of claim 37 wherein the polyethylene glycol diacrylate comprises a molecular weight between 700 and 800.

42. The system of claim 41 wherein the pentaerthyritol tetra 3(mercaptopropionate) is in a proportion ranging from about 0.31 to about 0.53 times the weight percent of the polyethylene glycol diacrylate present.

43. The system of claim 37 wherein the embolic material further comprises saline or other inert biocompatible materials.

44. The system of claim 31 wherein the embolic material has a first viscosity upon delivery into the perigraft space and is a solid after the embolic material has substantially cured.

45. A kit for depositing an embolic material in a perigraft space between an endovascular graft and a body lumen wall, the kit comprises:
a delivery device configured to access the perigraft space; and
an embolic material comprising polyethylene glycol diacrylate, pentaerthyritol tetra 3(mercaptopropionate), and a buffer.

46. The kit of claim 45 wherein the delivery device comprises a catheter.

47. The kit of claim 45 wherein the delivery device comprises a syringe and needle configured to percutaneously access the perigraft space.

48. The kit of claim 45 wherein the buffer comprises a glycylglycine buffer.

49. The kit of claim 48 wherein the glycylglycine buffer is present in a proportion ranging from about 5 to about 40 weight percent.

50. The kit of claim 45 wherein the polyethylene glycol diacrylate is present in a proportion ranging from about 50 to about 55 weight percent.

51. The kit of claim 45 wherein the polyethylene glycol diacrylate comprises a molecular weight between 700 and 800.

52. The kit of claim 51 wherein the pentaerthyritol tetra 3(mercaptopropionate) is present in a proportion ranging from about 0.31 to about 0.53 times the weight percent of the polyethylene glycol diacrylate present.

53. The kit of claim 45 further comprising an occlusion member that is configured to temporarily occlude the body lumen.

54. The kit of claim 53 wherein the occlusion member is an inflatable balloon.

55. The kit of claim 45 wherein the buffer comprises HEPES.
